(19) United States
(12) Patent Application Publication
(10) Pub. No.: US 2011/0044913 A1
(43) Pub. Date: Feb. 24, 2011

(54) 8-HYDROXY-5-[(1R)-1-HYDROXY-2-[[(1R)-2-(4-METHOXYPHENYL)-1-METHYLETHYL]AMINO]ETHYL]-2(1H)-QUINOLINONE HEMI-FUMARATE

(75) Inventors: Fausto Pivetti, Parma (IT); Emilio Lutero, Parma (IT)

Correspondence Address: OBLON, SPIVAK, MCCLELLAND MAIER & NEUSTADT, L.L.P., 1940 DUKE STREET, ALEXANDRIA, VA, 22314, US

(73) Assignee: Chiesi Farmaceutici S.p.A., Parma (IT)

(21) Appl. No.: 12/846,256

(22) Filed: Jul. 29, 2010

(30) Foreign Application Priority Data
Aug. 4, 2009 (EP) 09167207.1

(51) Int. Cl. A61K 9/12; C07D 215/26; A61K 31/4704; A61K 31/56; A61K 31/58; A61P 11/00; A61P 11/06

(52) U.S. Cl. 424/43; 546/157; 514/312; 514/171

(57) ABSTRACT

The invention relates to 8-hydroxy-5-[(1R)-1-hydroxy-2-[[(1R)-2-(4-methoxyphenyl)-1-methylethyl]-amino]ethyl]-2(1H)-quinolinone hemi-fumarate salt. The invention also relates to processes for its preparation, pharmaceutical compositions thereof, and to its use as a medicament.

### CROSS REFERENCES TO RELATED APPLICATIONS

This application claims priority to European Patent Application No. 09167207.1 filed on Aug. 4, 2009, which is incorporated herein by reference in its entirety.

### BACKGROUND OF THE INVENTION

1. Field of the Invention

The present invention relates to a novel 8-hydroxy-5-[(1R)-1-hydroxy-2-[[(1R)-2-(4-methoxyphenyl)-1-methylethyl]-amino]ethyl]-2(1H)-quinolinone salt. The present invention also relates to pharmaceutical compositions which contain such a salt, methods of making such a salt, and to uses of such a salt as a medicament.

2. Discussion of the Background

The pharmaceutically active compound 8-hydroxy-5-[(1R)-1-hydroxy-2-[[(1R)-2-(4-methoxyphenyl)-1-methylethyl]amino]ethyl]-2(1H)-quinolinone (I), also known as carmoterol, has been described in EP 0 147 719 as a bronchodilator having a potent beta₂-adrenoceptor stimulating action:

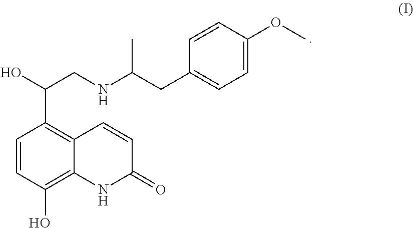

Two positions in the carmoterol structure may be protonated or deprotonated to form salts, i.e. the basic amino group and the acidic phenolic group to form ammonium salts and phenate salts, respectively.

Only carmoterol monohydrochloride salt on the amino group, referred to with the code TA 2005 or CHF 4226, has been widely investigated as a medicament for the treatment of respiratory disorders such as asthma and chronic obstructive pulmonary disease (COPD). However, the monohydrochloride salt can be obtained in an adequate level of chemical purity and crystallinity only by applying specific crystallization conditions, as reported in WO 2005/089760.

Moreover, said salt is presently under development as formulations for inhalation at very low single doses, ranging from 1 to 4 μg per actuation of the inhaler. Therefore, in the case of powder or suspension formulations, it is important that the drug disperses well and is uniformly distributed in the composition, since a lack of homogeneity, upon administration through the inhaler, could involve a risk of an over- or under-dosage, and hence be detrimental to the possibility of achieving a reproducible accuracy of the delivered dose.

So far, carmoterol monohydrochloride has been found difficult to be dispersed, due to the possible formation of agglomerates which cannot sufficiently be broken by the mixing energy.

Other ammonium salts have generically been disclosed in EP 0 147 719 and in other documents such as WO 2005/013994 and WO 2007/014673, but they have not been further characterized.

In view of the aforementioned problems, it would be advantageous to provide a carmoterol salt which is easy to obtain in a highly crystalline form as well as providing good handling qualities for pharmaceutical use, in particular in terms of dispersibility in a composition for inhalation.

### SUMMARY OF THE INVENTION

Accordingly, it is one object of the present invention to provide a novel salt of 8-hydroxy-5-[(1R)-1-hydroxy-2-[[(1R)-2-(4-methoxyphenyl)-1-methylethyl]amino]ethyl]-2(1H)-quinolinone (carmoterol).

It is another object of the present invention to provide a novel salt of 8-hydroxy-5-[(1R)-1-hydroxy-2-[[(1R)-2-(4-methoxyphenyl)-1-methylethyl]amino]ethyl]-2(1H)-quinolinone (carmoterol) which is easy to obtain in a highly crystalline form.

It is another object of the present invention to provide a novel salt of 8-hydroxy-5-[(1R)-1-hydroxy-2-[[(1R)-2-(4-methoxyphenyl)-1-methylethyl]amino]ethyl]-2(1H)-quinolinone (carmoterol) which has good handling properties.

It is another object of the present invention to provide a novel salt of 8-hydroxy-5-[(1R)-1-hydroxy-2-[[(1R)-2-(4-methoxyphenyl)-1-methylethyl]amino]ethyl]-2(1H)-quinolinone (carmoterol) which is easy to disperse in a composition for inhalation.

It is another object of the present invention to provide novel methods for making such a salt of 8-hydroxy-5-[(1R)-1-hydroxy-2-[[(1R)-2-(4-methoxyphenyl)-1-methylethyl]amino]ethyl]-2(1H)-quinolinone (carmoterol).

It is another object of the present invention to provide novel pharmaceutical compositions which contain such a salt of 8-hydroxy-5-[(1R)-1-hydroxy-2-[[(1R)-2-(4-methoxyphenyl)-1-methylethyl]amino]ethyl]-2(1H)-quinolinone (carmoterol).

It is another object of the present invention to provide novel methods of treating various conditions or diseases by administering such a salt of 8-hydroxy-5-[(1R)-1-hydroxy-2-[[(1R)-2-(4-methoxyphenyl)-1-methylethyl]amino]ethyl]-2(1H)-quinolinone (carmoterol).

These and other objects, which will become apparent during the following detailed description, have been achieved by the inventors' discovery of a carmoterol salt meeting said requirements, in particular the 8-hydroxy-5-[(1R)-1-hydroxy-2-[[(1R)-2-(4-methoxyphenyl)-1-methylethyl]amino]ethyl]-2(1H)-quinolinone (carmoterol) hemi-fumarate salt.

Thus, the present invention provides the 8-hydroxy-5-[(1R)-1-hydroxy-2-[[(1R)-2-(4-methoxyphenyl)-1-methylethyl]amino]ethyl]-2(1H)-quinolinone (carmoterol) hemi-fumarate salt.

The salt of the present invention may be produced by crystallization from appropriate solvents and conditions. It is characterized by specific peaks in the X-ray powder diffraction (XRPD) pattern. Accordingly, the present invention also provides processes for the preparation of said salt by crystallization from appropriate solvents.

In another aspect, the present invention provides pharmaceutical compositions which comprise the salt of carmoterol, and a pharmaceutically acceptable carrier.

The invention also provides the use of the salt of the invention as a medicament.

In a further aspect, the present invention provides the use of the salt of the invention for the prevention and/or treatment of an inflammatory or obstructive respiratory disease such as asthma or chronic obstructive pulmonary disease (COPD).

Moreover, the present invention provides the use of the salt of the invention in the manufacture of a medicament for the prevention and/or treatment of an inflammatory or obstructive respiratory disease such as asthma or chronic obstructive pulmonary disease (COPD).

In yet another aspect, the present invention provides methods for preventing and/or treating an inflammatory or obstructive respiratory disease such as asthma or chronic obstructive pulmonary disease (COPD), the method comprising administering an effective amount of the salt of the invention to patients in need thereof.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows the X-ray diffraction pattern (XRDP) of carmoterol hemi-fumarate.

FIG. 2 shows the IR spectrum of carmoterol hemi-fumarate.

FIG. 3 shows the Raman spectrum of carmoterol hemi-fumarate.

### DETAILED DESCRIPTION OF THE PREFERRED EMBODIMENTS

Unless defined otherwise, all technical and scientific terms used herein have the same meanings as commonly understood by the skilled in the art.

The term “hemi-fumarate” refers to the salt of carmoterol with fumaric acid in the 2:1 stoichiometric ratio of carmoterol to fumaric acid.

“Fumarate”, refers to the salt with a stoichiometrically equimolar amount of fumaric acid.

The term “amorphous” describes a non-ordered solid state characterized by a diffused X-ray powder diffraction with no sharp peaks.

An effective amount of a compound for treating a particular disease is an amount that is sufficient to ameliorate, or in some manner reduce the symptoms associated with the disease.

The term “high level of chemical purity” refers to a compound wherein the total amount of readily detectable impurities as determined by standard methods of analysis, such as high performance liquid chromatography (HPLC), is less than 5%, advantageously less than 2.5%, preferably less than 1.0, more preferably less than 0.5% w/w.

The term “high level of crystallinity” refers to a polymorph wherein the percentage of crystallinity is equal to or higher than 85% as determined by standard methods of analysis, such as X-ray powder diffraction or microcalorimetry.

The term “good flowability” refers to a formulation that is easy handled during the manufacturing process and is capable of ensuring an accurate and reproducible delivery of the therapeutically effective dose.

Flow characteristics can be evaluated by measuring the Carr's index; a Carr's index of less than 25 is usually considered to define good flow characteristics. Otherwise, it can be measured using suitable apparatus such as the Flodex tester.

The term “good homogeneity” refers to a formulation wherein, upon mixing, the content of active material contained in multiple different samples collected throughout the batch is always within 85 and 115 by weight %.

The term “respirable fraction” refers to an index of the percentage of active particles which would reach the deep lungs in a patient.

The respirable fraction, also termed fine particle fraction, is evaluated using a suitable in vitro apparata such as Multistage Cascade Impactor or Multi Stage Liquid Impinger (MLSI) according to procedures reported in common Pharmacopoeias. It is calculated by the ratio between the respirable dose and the delivered dose.

The delivered dose is calculated from the cumulative deposition in the apparatus, while the respirable dose (fine particle dose) is calculated from the deposition on Stages 3 (S3) to filter (AF) corresponding to particles ≦5.0 micron.

As a thumb rule, a respirable fraction higher than 30% is an index of good inhalatory performances.

“Interactive ordered mixture” refers to a powder wherein the cohesive micronized active ingredient is mixed with free-flowing coarser carrier particles on which surface the micronized particles adhere.

It has been found that carmoterol hemi-fumarate salt can be easily and homogeneously dispersed in a carrier for a powder formulation providing a good uniformity of distribution of the active particles, and hence, an adequate accuracy of the metered dose. Moreover, contrary to the fumarate salt and other salts, the hemi-fumarate salt can be prepared in a highly crystalline form, is readily isolable and exhibits favourable filtration characteristics. The hemi-fumarate salt is also non-hygroscopic and chemically and physically stable in different conditions of humidity and temperature.

The carmoterol hemi-fumarate of the present invention is preferably anhydrous, but the invention also provides solvates and hydrates of said salt.

In one embodiment of the present invention, carmoterol hemi-fumarate has a degree of crystallinity equal to or higher than 85%, advantageously equal to or higher than 90, more advantageously of at least 95%, preferably of at least 98%.

The hemi-fumarate salt may be characterized by analytical methods such as X-ray powder diffraction (XRPD), Infra-Red (IR) and Raman spectroscopy, or by its melting point measured by melting point apparatus or Differential Scanning Calorimetry (DSC).

In one embodiment, the hemi-fumarate salt exhibits the characteristics XRPD peaks expressed in degrees 2 (2-theta) at approximately the diffraction angles reported in Table 1.

TABLE 1
Diffraction Angle (° 2θ)

5.99
11.53
11.96
12.21
14.19
14.32
15.26
18.01
18.29
18.79
19.01
20.61
20.85
21.30
21.95
22.24
23.17
23.44
23.58
24.17
24.42
26.93

The term “approximately” means that there is an uncertainty in the measurements of the degrees 2θ of ±0.2.

In another embodiment, the hemi-fumarate salt has diffraction peaks at approximately the 2θ angles, measured by Cu Kα radiation, shown in Table 2.

TABLE 2
Degrees 2-theta

5.99
11.53
11.96
20.85
21.30
21.95
23.58

In another embodiment, the hemi-fumarate salt has diffraction peaks at approximately the 2θ angles, measured by Cu Kα radiation, shown in Table 3.

TABLE 3
Degrees 2-theta

5.99
11.53
11.96
14.3
19.01
20.85
21.30
21.95
23.44
23.58
24.42

In another embodiment, the hemi-fumarate salt is characterized by a XRPD pattern comprising characteristic peaks with approximate 2θ values as indicated in Table 4, and with relative intensities deviating by no more than ±30%, preferably no more than ±10%, from the values given in Table 4.

TABLE 4

| Degrees 2-theta | Relative intensity (%) |
|---|---|
| 5.99 | 100 |
| 11.53 | 50 |
| 11.96 | 29 |
| 14.3 | 11 |
| 19.01 | 13 |
| 20.85 | 23 |
| 21.30 | 22 |
| 21.95 | 26 |
| 23.44 | 16 |
| 23.58 | 24 |
| 24.42 | 18 |

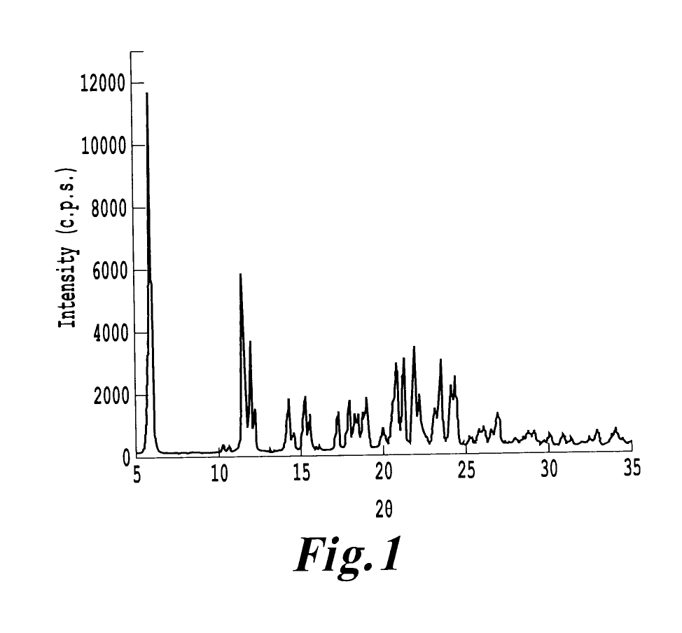

The salt of the present invention may have an XRPD pattern having characteristic peaks and relative intensities substantially as illustrated in FIG. 1.

The salt of the present invention may be further characterized by an IR spectrum having the main peaks at the following wave numbers (cm⁻¹) with the relative intensities between brackets:

3415-2135 (broad), 1644 (strong), 1558 (strong), 1353 (strong), 1248 (strong), 872-614 (medium).

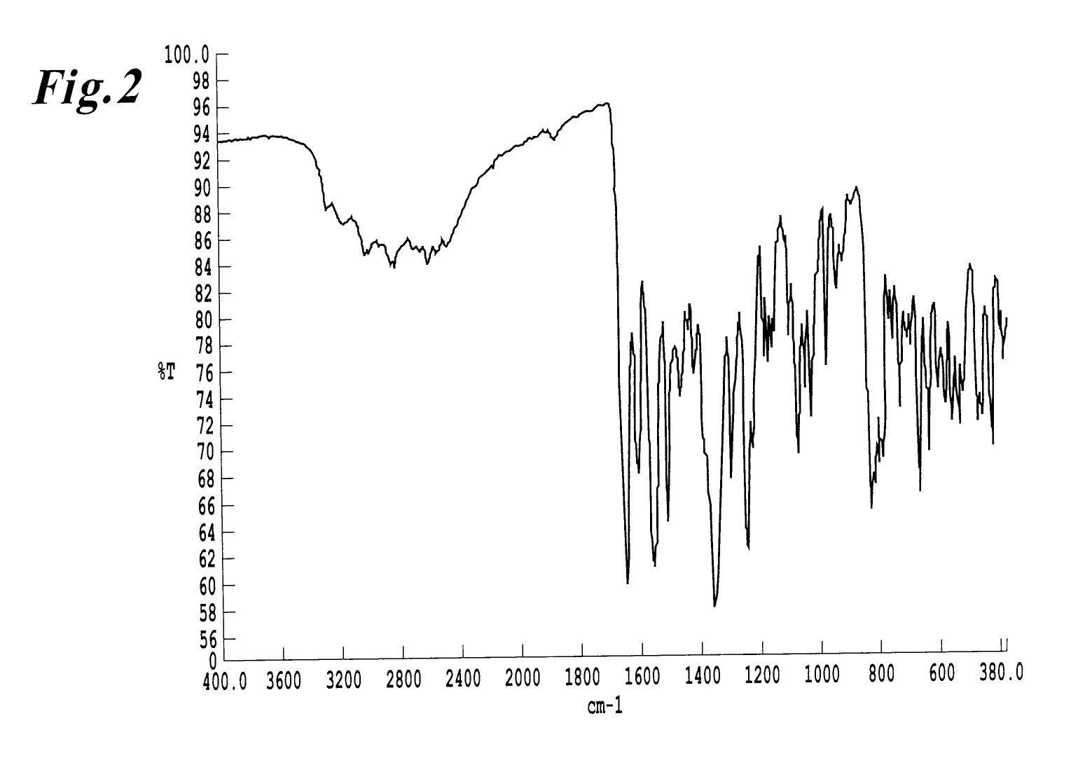

The salt of the present invention may also be defined as having the IR spectrum having characteristic peaks and relative intensities substantially as illustrated in FIG. 2.

Raman spectroscopy may also be useful for characterizing the salt of the present invention having the main peaks at the following wave numbers (cm⁻¹) with the relative intensities between brackets:

317-2780 (broad), 1650 (medium), 1612 (medium), 1410 (strong), 1330 (very strong).

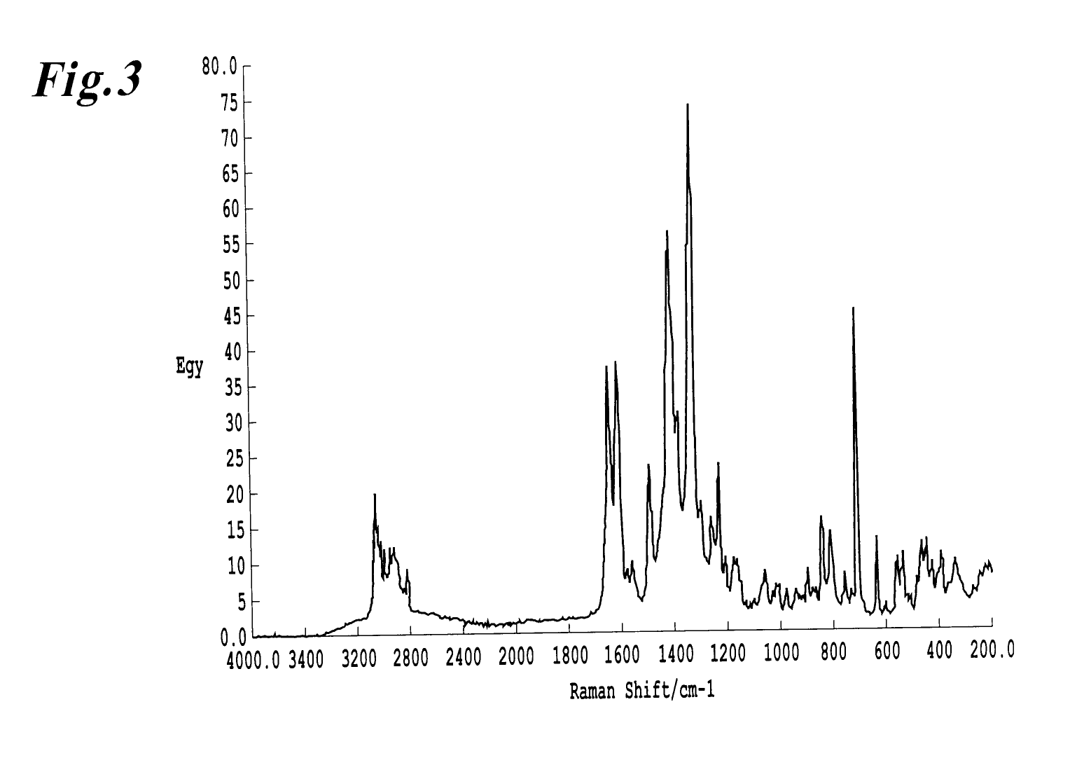

The salt of the present invention may have the Raman spectrum having characteristic peaks and relative intensities substantially as illustrated in FIG. 3.

The hemi-fumarate salt of carmoterol has a melting range comprised between 191 and 194° C. as measured by DSC.

The salt of the present invention shows a high level of chemical purity and enantiomeric purity. Advantageously, the chemical purity is higher than 90%, preferably higher 95%, more preferably higher than 98% as determined by High-Pressure Liquid Chromatography (HPLC), while the enantiomeric purity is higher than 95%, preferably higher than 98% as measured by capillary zone electrophoresis.

The solubility of the salt of the present invention in different solvents expressed as %, w/v, is reported in Table 5.

TABLE 5

| Salt | Water | Ethanol | Acetone | Vertrel XF⁽¹⁾ |
|---|---|---|---|---|
| Carmoterol hemi-fumarate | 1.36 | 0.27 | 0.008 | <l.o.d.⁽²⁾ |

⁽¹⁾Vertrel XF ® is the trade name for 2,3-dihydodecafluoropentane which is used as a solvent to simulate the properties of common HFA propellants like HFA134a as it can easily be handled due to its higher boiling point (55° C.)
⁽²⁾l.o.d.: limit of detection.

The present invention also provides a process for the preparation of the salt of the invention, comprising the following steps:

(i) dissolving carmoterol free base in a suitable solvent, optionally under heating;

(ii) dissolving the proper amount of fumaric acid in the minimum possible volume of a suitable solvent;

(iii) adding the solution of the acid to the carmoterol solution;

(iv) cooling and optionally concentrating the obtained solution;

(v) adding a suitable volume of another solvent to favor the crystallization; and

(vi) isolating the precipitate from the solution.

A preferred solvent for both carmoterol base and fumaric acid is ethanol. A suitable solvent for favoring the crystallization is isopropyl ether.

The amount of solvents may be adjusted according to the known methods. The isolation may also be carried out according known, e.g. by filtration.

Carmoterol hemi-fumarate may be formulated in any convenient way. The invention also provides pharmaceutical compositions comprising the salt of the present invention, and one or more pharmaceutically acceptable carriers or excipients such as diluents, wetting agents, emulsifying agents, binders, coatings, fillers, glidants, lubricants, disintegrants, preservatives, stabilizers, surfactants, pH buffering substances, flavouring agents and the like. A comprehensive guidance on pharmaceutical excipients is given in Remington's Pharmaceutical Sciences Handbook, XVII Ed. Mack Pub., N.Y., U.S.A., which is incorporated herein by reference in its entirety.

The salt of the present invention may be formulated for oral, buccal, topical, parenteral, vaginal, rectal or inhalation administration, preferably for inhalation administration.

Inhalable compositions include inhalable dry powders, pressurized metered dose inhalers (pMDI) formulations containing propellants such as hydrofluoroalkanes (HFAs) or propellant-free inhalable solution or suspension formulations.

The present invention preferably provides a powder formulation for inhalation, comprising the salt of the present invention, in the form of interactive ordered mixture wherein the carrier particles have a mass median diameter (MMD) higher than 50 microns, preferably higher than 90 microns.

The carrier particles may comprise one or more pharmacologically inert physiologically acceptable excipients such as crystalline sugars, preferably lactose, most preferably alpha-lactose monohydrate.

More advantageously, said formulation comprises a fraction of coarse particles made of alpha-lactose monohydrate, said particles having a MMD higher than 90 microns, preferably the mass diameter (MD) comprised between 50 microns and 500 microns, more preferably between 150 and 400 microns, even more preferably between 210 and 355 microns.

Preferably, said powder formulation further comprises a fraction of microparticles obtained by co-milling having a MMD lower than 35 microns, and comprising particles of alpha-lactose monohydrate and an additive material selected from anti-adherents such as the amino acids leucine and isoleucine or lubricants such as magnesium stearate; sodium stearyl fumarate stearyl alcohol, stearic acid and sucrose monopalmitate.

More preferably, said powder formulation comprises a fraction of microparticles having a MMD lower than 15 microns, preferably lower than 10 microns, comprising particles of alpha-lactose monohydrate and particles of magnesium stearate.

Due to its dispersibility and solubility characteristics, the salt of the present invention may also turn out to be particularly suitable for providing pMDI formulations wherein the active ingredient is in suspension since carmoterol hemi-fumarate is practically insoluble in HFA propellants.

The salt of the present invention in the aforementioned powder and pMDI formulations is advantageously used in micronized form. Advantageously, at least 90% of the micronized particles have a volume diameter lower than 10 microns, more advantageously equal to or lower than 8 microns. Preferably the micronized particles have a particle size distribution wherein: i) no more than 10% of the particles have a volume diameter [d(v,0.1)] lower than 0.7 microns, preferably lower than 0.8 microns; ii) no more than 50% of particles have a volume diameter [d(v,0.5)] lower than 1.7 microns, preferably comprised between 2 and 4 microns; and iii) at least 90% of the particles have a volume diameter equal to or lower than 8 microns, preferably lower than 6 microns.

The dosage of the salt of the invention can vary within wide limits depending on the nature of the disease to be treated, the type of patient and the mode of administration and will be determined according to known methods. Upon inhalation, a typical daily dosage is within the range of 1 μg to 8 μg preferably of 2 to 4 μg administered once or twice a day.

The salt of the present invention may be used in combination with one or more other therapeutic agents, preferably those currently used in the treatment of respiratory disorders, e.g. corticosteroids such as budesonide and its epimers, beclometasone dipropionate, triamcinolone acetonide, fluticasone propionate, flunisolide, mometasone furoate, rofleponide and ciclesonide, anticholinergic or antimuscarinic agents such as ipratropium bromide, oxytropium bromide, tiotropium bromide, glycopyrrolate bromide and phosphodiesterase-4 (PDE-4) inhibitors such as roflumilast. The combination with budesonide is preferred.

Carmoterol hemi-fumarate may be used for the preparation of a medicament for any disease or condition in which it is therapeutically effective.

In view of its beta₂-adrenoceptor stimulating activity, the salt of the present invention is useful in the relaxation of bronchial smooth muscle and the relief of bronchoconstriction. Relief of bronchoconstriction can be measured in models such as the in vivo guinea pigs model (see, Kikkawa et al., Biol. Pharm. Bull., 1994, 17(8), 1047-1052) and analogous models.

Therefore, administration of the salt of the present invention may be indicated for the prevention and/or treatment of mild, moderate or severe acute or chronic symptoms or for prophylactic treatment of obstructive respiratory diseases such as asthma and chronic obstructive pulmonary disease (COPD). Other respiratory disorders characterized by obstruction of the peripheral airways as a result of inflammation and presence of mucus such as chronic obstructive bronchiolitis and chronic bronchitis may also benefit from their use.

Other features of the invention will become apparent in the course of the following descriptions of exemplary embodiments which are given for illustration of the invention and are not intended to be limiting thereof.

### EXAMPLES

### Example 1

### Preparation of Carmoterol Hemi-Fumarate

In a 1 l flask, carmoterol free base (20 g, 0.054 mol) is dissolved in 400 ml of refluxing ethanol 95% v/v. Fumaric acid (3.16 g, 0.027 mol) is dissolved in hot ethanol and added to the solution. The mixture is concentrated under vacuum until approximately 200 ml of ethanol are distilled off, then 100 ml of diisopropyl ether is slowly added to the solution at 40 to 50° C. After cooling, the salt in form of a solid crystallizes; it is filtered, triturated in 100 ml of ethyl ether, filtered again and dried at 40° C. under vacuum for 15 hours. The crude product (17 g) is dissolved in 425 ml of refluxing ethanol, the solution is cooled to 10° C., stirred at 10° C. for 60 minutes, then the crystallized solid is filtered, and washed with 20 ml of ethanol.

Yield: 13.5 g (58.3%);

Assay (HPLC): >99.5%;

Enantiomeric purity (CZE): 100%

Melting range (DSC): 191-194° C.

### Example 2

### Solid State Characterization

The hemi-fumarate salt, obtained as a white powder, is analyzed in the solid state, e.g. by XRPD, IR and Raman spectroscopy.

### 1. X-Ray Powder Diffraction (XRPD).

The XRPD analysis is carried out on a Thermo Electron X-ray powder diffractometer using Cu Kα radiation. A theta-two theta continuous scan from 5 degrees 2 theta to 35 degrees 2 theta is used. The sample is prepared for analysis by placing it in a quartz sample holder. The XPRD pattern is shown in FIG. 1.

### 2. IR Spectrum.

The IR spectrum is acquired on a Perkin-Elmer FT-IR spectrophotometer Spectrum 400 (scan range 4000-400 cm⁻¹). It is reported in FIG. 2.

### 3. Raman Spectrum.

The Raman spectrum is obtained with a Perkin-Elmer. FT-Raman spectrometer System 2000R supplied with radiation of 1064 nm and a InGaAs detector (scan range 4000-200 cm⁻¹). It is reported in FIG. 3.

### Example 3

### Preparation of Micronized Carmoterol Hemi-Fumarate

A batch of formoterol fumarate dihydrate is milled in a nitrogen fluid jet mill apparatus MC 50 (JET Pharma S.A). The particle size distribution and other physico-chemical characteristics are reported in Table 6.

TABLE 6
Carmoterol hemi-fumarate

| Particle size (μm) | |
|---|---|
| d (v, 0.1) | 0.79 |
| d (v, 0.5) | 3.73 |
| d (v, 0.9) | 7.58 |
| Assay (HPLC) | >99.5% |
| PXRD | crystalline |

As it can be appreciated from Table 6, upon milling, the salt of the present invention remains crystalline and its chemical purity is not altered. The batch is then added to a carrier made of coarser particles.

The homogeneous dispersion of the active ingredient in the powder, and the absence of aggregates of active particles is established using a Near Infrared spectrophotometer equipped with a microscopy imaging system (Near Imaging).

The salt of the present invention in micronized form uniformly disperses into the carrier and after 180 minute of mixing s no agglomerates are observed.

### Example 4

### Exemplary Inhalable Dry Powder Formulation Comprising Carmoterol Hemi-Fumarate

The formulation is prepared according to the teaching of WO 01/78693. The composition is reported in Table 7.

TABLE 7

| | Amounts | | |
|---|---|---|---|
| | Per shot of the inhaler | | Single dose |
| Components | mg | % | μg |
| Carmoterol hemi-fumarate | 0.001 | 0.01 | 1 |
| Alpha-lactose monohydrate | 8.999 | 89.99 | |
| 212-355 μm | | | |
| microparticles of alpha-lactose | 1.00 | 10.0 | |
| monohydrate and magnesium | | | |
| stearate obtained by co-milling | | | |
| Total weight | 10 | | |

### Example 5

### Characterization of the Formulation of Example 4

The technological characteristics and aerosol performances of the formulation of Example 4 are evaluated after loading in a multidose dry powder inhaler. The uniformity of distribution of the active ingredient is evaluated by taking 10 samples, each equivalent to about from one to three doses, from different parts of the blend.

The flowability is evaluated from the Carr's index by determining the poured density (dv) and the tapped density (ds) as follows. Powder mixtures (20 g) are poured into a glass graduated cylinder and dv was calculated dividing the weight by the volume; ds is calculated from the volume obtained after tapping the powder mixture 500 times using a commercially available apparatus.

The flowability is evaluated from the Carr's index calculated according to the following formula:

Carr's index(%)=(ds−dv)/ds×100

The flowability properties are also determined by using a Flodex tester according to the method reported in the European Pharmacopeia 4^{th} Ed, 2002, paragraph 2.9.16. The powder mixture is poured into a dry funnel equipped with an orifice of suitable diameter that is blocked by suitable mean. The bottom opening of the funnel is unblocked and the time needed for the entire sample to flow out of the funnel recorded. The flowability is expressed in seconds related to 100 g of sample (s/100 g). The emptying index is defined as the percentage of the powder flown through a metered diameter hole to the tested amount of the sample.

The evaluation of the aerosol performance is carried out using a Multi Stage Liquid Impinger (MSLI) apparatus (Apparatus C) according to the conditions reported in the Eur Ph 4^{th} Ed 2002, par 2.9.18, pages 213-219. After aerosolization of 10 doses, the MSLI apparatus is disassembled and the amounts of drug deposited in the stages are recovered by washing with a solvent mixture and then quantified by HPLC. The following parameters are calculated: i) the delivered dose which is the amount of drug delivered from the device recovered in the impactor; ii) the fine particle dose (FPD) which is the amount of delivered dose recovered below 5.0 microns; iii) the fine particle fraction (FPF) which is the percentage of the fine particle dose relative to the delivered dose reaching the stage 2 of TSI. The results are reported in Table 8.

TABLE 8
Technological characteristics and aerosol performances.

| | |
|---|---|
| Poured density (g/ml)) | 0.65 |
| Tapped density (g/ml) | 0.74 |
| Carr's index (%) | 12.2 |
| Flow rate through 4 mm Ø (s/100 g) | 138 |
| Emptying index (%) | 98.5 |
| Uniformity of distribution of the active ingredient (%) | 105 |
| Delivered dose (μg) | 1.05 |
| FPD (μg) | 0.53 |
| FPF (%) | 50.1 |

The powder formulation comprising the salt of the present invention shows a good uniformity of distribution of the active ingredient with an average content uniformity of 105% and no sample with a value outside the limits 85 and 115%. The flowability is also very good as demonstrated by the low Can's index and by the emptying index near to 100%. Moreover, the aerosol performances of the formulation are very good with about than 50% of respirable fraction.

Where a numerical limit or range is stated herein, the endpoints are included. Also, all values and subranges within a numerical limit or range are specifically included as if explicitly written out.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. 8-hydroxy-5-[(1R)-1-hydroxy-2-[[(1R)-2-(4-methoxyphenyl)-1-methylethyl]amino]ethyl]-2(1H)-quinolinone hemi-fumarate salt.

2. Crystalline 8-hydroxy-5-[(1R)-1-hydroxy-2-[[(1R)-2-(4-methoxyphenyl)-1-methylethyl]amino]ethyl]-2(1H)-quinolinone hemi-fumarate salt.

3. The salt according to claim 2, which has X-ray powder diffraction pattern peaks expressed in angle 2-theta at approximately: 5.99; 11.53; 11.96; 20.85; 21.30; 21.95; and 23.58.

4. The salt according to claim 2, which has X-ray powder diffraction pattern peaks expressed in angle 2-theta at approximately: 5.99; 11.53; 11.96; 14.3; 19.01; 20.85; 21.30; 21.95; 23.44; 23.58; and 24.42.

5. The salt according to claim 2, which has X-ray powder diffraction pattern peaks expressed in angle 2-theta at approximately: 5.99; 11.53; 11.96; 14.19; 14.32; 15.26; 18.01; 18.29; 18.79; 19.01; 20.61; 20.85; 21.30; 21.95; 22.24; 23.17; 23.44; 23.58; 24.17; 24.42; 26.93.

6. A process for preparing the salt of claim 1, comprising:
(i) dissolving carmoterol free base in a first solvent, to obtain a first solution;
(ii) dissolving a proper amount of the fumaric acid in a second solvent, to obtain a second solution;
(iii) adding said second solution to said first solution, to obtain a third solution;
(iv) cooling and optionally concentrating, said third solution;
(v) adding a volume of another solvent to favor crystallization of 8-hydroxy-5-[(1R)-1-hydroxy-2-[[(1R)-2-(4-methoxyphenyl)-1-methylethyl]amino]ethyl]-2(1H)-quinolinone hemi-fumarate; and
(vi) isolating a precipitate of 8-hydroxy-5-[(1R)-1-hydroxy-2-[[(1R)-2-(4-methoxyphenyl)-1-methylethyl]amino]ethyl]-2(1H)-quinolinone hemi-fumarate.

7. A method according to claim 6, wherein said fumaric acid is dissolved in a minimum possible volume of said second solvent;

8. A pharmaceutical composition, comprising the salt according to claim 1 and a pharmaceutically acceptable carrier.

9. A pharmaceutical composition according to claim 8, further comprising a therapeutic agent selected from corticosteroids, anticholinergic or antimuscarinic agents and phosphodiesterase-4 (PDE-4) inhibitors.

10. A pharmaceutical composition according to claim 9, which comprises budesonide.

11. A pharmaceutical composition according to claim 8, which is in the form of an inhalable aerosol comprising a propellant.

12. A pharmaceutical composition according to claim 9, which is in the form of an inhalable aerosol comprising a propellant.

13. A pharmaceutical composition according to claim 10, which is in the form of an inhalable aerosol comprising a propellant.

14. A pharmaceutical composition according to claim 8, which is in the form of an inhalable dry powder.

15. A pharmaceutical composition according to claim 9, which is in the form of an inhalable dry powder.

16. A pharmaceutical composition according to claim 10, which is in the form of an inhalable dry powder.

17. A method of treating and/or preventing a respiratory disease, comprising administering to a subject in need thereof a salt according to claim 1.

18. A method according to claim 17, wherein said respiratory disease is an inflammatory or obstructive respiratory disease.

19. A method according to claim 17, wherein said respiratory disease is asthma or chronic obstructive pulmonary disease.
